(19) United States
(12) Patent Application Publication
(10) Pub. No.: US 2021/0251479 A1
(43) Pub. Date: Aug. 19, 2021

(54) AUTOMATED DETECTION IN CERVICAL IMAGING

(71) Applicant: MOBILEODT, LTD., Tel Aviv (IL)

(72) Inventors: David LEVITZ, Tel Aviv (IL); Amir Shlomo BERNAT, Moshav Mazor (IL)

(21) Appl. No.: 16/973,224

(22) PCT Filed: Jun. 13, 2019
(86) PCT No.: PCT/IL2019/050669

§ 371 Date: Dec. 8, 2020

Related U.S. Application Data
(60) Provisional application No. 62/684,322, filed on Jun. 13, 2018.

(51) Int. Cl. A61B 1/303; A61B 1/00; G06N 3/08

(52) U.S. Cl. CPC A61B 1/303; G06N 3/08; A61B 1/00055; A61B 1/00009

(57) ABSTRACT

A method comprising capturing at least one image of a cervical tissue in-vivo; identifying a region of interest (ROI) in said cervical tissue within said at least one image; detecting at least a portion of a vaginal speculum within said at least one image; and determining a position of said portion of said vaginal speculum relative to said ROI.

### CROSS-REFERENCE TO RELATED APPLICATIONS

This application claims the benefit of priority to U.S. Provisional Patent Application No. 62/684,322, filed Jun. 13, 2018, entitled “AUTOMATED DETECTION IN CERVICAL IMAGING,” the contents of which are incorporated herein by reference in their entirety.

### FIELD OF THE INVENTION

The invention relates generally to medical imaging systems.

### BACKGROUND

The main target of cervical cancer screening is to identify cervical intraepithelial neoplasia (CIN), a premalignant lesion that can progress to cervical cancer if left untreated. Cervicography, or digital imaging of the cervix can be used as an alternative method to Papanicolaou (Pap) smear test and HPV testing, especially in low resource settings, where there is inadequate cancer screening infrastructure, poorly-trained medical staff, and highly-variable detection practice.

Digital cervicography can improve the efficacy of cervical cancer screening by enabling automatic diagnosis applications in combination with remote consultations. However, there are various challenges for the extraction of useful information from cervix images, which are sometimes related to failure to follow correct cervicography procedures. One such issue is related to poor speculum positioning which can occlude parts of the cervix in the images and make it difficult to visualize the entire cervix. In addition, vaginal walls which become lax can further obstruct portions of the cervical area.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in accordance with an embodiment, a method comprising capturing at least one image of a cervical tissue in-vivo; identifying a transformation zone of said cervical tissue within said at least one image; detecting at least a portion of a vaginal speculum within said at least one image; and determining a position of said portion of said vaginal speculum relative to said transformation zone (TZ).

In some embodiments, the method further comprises issuing an alert when said determining indicates that said portion of said vaginal speculum obstructs, at least in part, said TZ in said at least one image, wherein said alert directs a clinician to reposition said vaginal speculum.

In some embodiments, the method further comprises repeating iteratively said detecting, determining, and issuing until said determining indicates that said portion of said vaginal speculum does not obstruct said TZ in said at least one image.

In some embodiments, the method further comprises capturing one or more images upon said indicating.

In some embodiments, said identifying comprises first identifying, in said at least one image, boundaries of said cervical tissue.

In some embodiments, said identifying is based, at least in part, on at least one of one of cervical tissue color and cervical surface texture.

In some embodiments, said identifying is based, at least in part, on executing one or more machine learning algorithms selected from the group consisting of convolutional neural network (CNN) classifiers and support vector machine (SVM) classifiers.

In some embodiments, said detecting is based, at least in part, on one or more methods of feature extraction, wherein said feature is an arch-like end portion of a blade of said vaginal speculum.

In some embodiments, said determining is based, at least in part, on a comparison of focus scores of (i) pixels in a region of said at least one image associated with said at least a portion of said vaginal speculum, and (ii) pixels in another region of said at least one image associated with said TZ.

In some embodiments, said determining is based, at least in part, on a morphologically dilated version of a region of said at least one image, wherein said region is associated with said at least a portion of said vaginal speculum.

In some embodiments, the method further comprises issuing an alert to direct a focus of the capturing to the cervix if the focus of the at least one image is determined to be on a vulva.

There is also provided, in accordance with an embodiment, a method comprising capturing at least one image of a cervical tissue in-vivo; identifying a transformation zone of said cervical tissue within said at least one image; detecting at least a portion of a vaginal wall within said at least one image; and determining a position of said portion of said vaginal wall relative to said transformation zone (TZ).

In some embodiments, the method further comprises issuing an alert when said determining indicates that said portion of said vaginal wall obstructs, at least in part, said TZ in said at least one image, wherein said alert directs a clinician to open said vaginal wall.

In some embodiments, the method further comprises repeating iteratively said detecting, determining, and issuing until said determining indicates that said portion of said vaginal wall does not obstruct said TZ in said at least one image.

In some embodiments, the method further comprises transmitting an image stream of said cervical tissue upon said indicating.

In some embodiments, the method further comprises identifying a medical accessory appearing in said image stream, wherein said identifying causes a countdown to begin; repeating iteratively said detecting, determining, and issuing until said determining indicates that said portion of said vaginal wall does not obstruct said TZ in said image stream; and capturing one or more images from said image stream upon the expiration of the countdown.

In some embodiments, the medical accessory is used for applying a contrast agent to the body tissue, wherein a duration of said countdown is determined at least in part based on the type of said contrast agent.

In some embodiments, said identifying comprises first identifying, in said at least one image, boundaries of said cervical tissue.

In some embodiments, said identifying is based, at least in part, on at least one of cervical tissue color and cervical surface texture.

In some embodiments, said identifying is based, at least in part, on executing one or more machine learning algorithms selected form the group consisting of convolutional neural network (CNN) classifiers and support vector machine (SVM) classifiers.

In some embodiments, said detecting is based, at least in part, on at least one of vaginal wall tissue color and vaginal surface texture.

In some embodiments, said detecting is based, at least in part, on one or more methods of feature extraction, wherein said feature is a ridges patterns of the surface of the vaginal wall.

In some embodiments, said determining is based, at least in part, on comparison of focus scores of (i) pixels in a region of said at least one image associated with said at least a portion of said vaginal wall, and (ii) pixels in another region of said at least one image associated with a central area of said TZ.

In some embodiments, said determining is based, at least in part, on a shape of a perimeter of said TZ.

There is further provided, in accordance with an embodiment, a system comprising at least one hardware processor; and a non-transitory computer-readable storage medium having stored thereon program instructions, the program instructions executable by the at least one hardware processor to operate an imaging device to capture at least one image of a cervical tissue in-vivo, identify a transformation zone of said cervical tissue within said at least one image, detect at least a portion of a vaginal speculum within said at least one image, and determine a position of said portion of said vaginal speculum relative to said transformation zone (TZ).

There is further provided, in accordance with an embodiment, a computer program product comprising a non-transitory computer-readable storage medium having program instructions embodied therewith, the program instructions executable by at least one hardware processor to operate an imaging device to capture at least one image of a cervical tissue in-vivo; identify a transformation zone of said cervical tissue within said at least one image; detect at least a portion of a vaginal speculum within said at least one image; and determine a position of said portion of said vaginal speculum relative to said transformation zone (TZ).

In some embodiments, said instructions further comprise issuing an alert when said determining indicates that said portion of said vaginal speculum obstructs, at least in part, said TZ in said at least one image, wherein said alert directs a clinician to reposition said vaginal speculum.

In some embodiments, said instructions further comprise repeating iteratively said detecting, determining, and issuing until said determining indicates that said portion of said vaginal speculum does not obstruct said TZ in said at least one image.

In some embodiments, said instructions further comprise operating said imaging device to capture one or more images upon said indicating.

In some embodiments, said identifying comprises first identifying, in said at least one image, boundaries of said cervical tissue.

In some embodiments, said identifying is based, at least in part, on at least one of one of cervical tissue color and cervical surface texture.

In some embodiments, said identifying is based, at least in part, on executing one or more machine learning algorithms selected from the group consisting of convolutional neural network (CNN) classifiers and support vector machine (SVM) classifiers.

In some embodiments, said detecting is based, at least in part, on one or more methods of feature extraction, wherein said feature is an arch-like end portion of a blade of said vaginal speculum.

In some embodiments, said determining is based, at least in part, on a comparison of focus scores of (i) pixels in a region of said at least one image associated with said at least a portion of said vaginal speculum, and (ii) pixels in another region of said at least one image associated with said TZ.

In some embodiments, said determining is based, at least in part, on a morphologically dilated version of a region of said at least one image, wherein said region is associated with said at least a portion of said vaginal speculum.

In some embodiments, said instructions further comprise issuing an alert to direct a focus of the capturing to the cervix if the focus of the imaging device is determined to be on a vulva.

There is further provided, in accordance with an embodiment, a system comprising at least one hardware processor; and a non-transitory computer-readable storage medium having stored thereon program instructions, the program instructions executable by the at least one hardware processor to operate an imaging device to capturing at least one image of a cervical tissue in-vivo, identify a transformation zone of said cervical tissue within said at least one image, detect at least a portion of a vaginal wall within said at least one image, and determine a position of said portion of said vaginal wall relative to said transformation zone (TZ).

There is further provided, in accordance with an embodiment, a computer program product comprising a non-transitory computer-readable storage medium having program code embodied therewith, the program code executable by at least one hardware processor to operate an imaging device to capturing at least one image of a cervical tissue in-vivo; identify a transformation zone of said cervical tissue within said at least one image; detect at least a portion of a vaginal wall within said at least one image; and determine a position of said portion of said vaginal wall relative to said transformation zone (TZ).

In some embodiments, said instructions further comprise issuing an alert when said determining indicates that said portion of said vaginal wall obstructs, at least in part, said TZ in said at least one image, wherein said alert directs a clinician to open said vaginal wall.

In some embodiments, said instructions further comprise repeating iteratively said detecting, determining, and issuing until said determining indicates that said portion of said vaginal wall does not obstruct said TZ in said at least one image.

In some embodiments, said instructions further comprise operating said imaging device to transmit an image stream of said cervical tissue upon said indicating.

In some embodiments, said instructions further comprise identifying a medical accessory appearing in said image stream, wherein said identifying causes a countdown to begin; repeating iteratively said detecting, determining, and issuing until said determining indicates that said portion of said vaginal wall does not obstruct said TZ in said image stream; and operating said imaging device to capture one or more images from said image stream upon the expiration of the countdown.

In some embodiments, the medical accessory is used for applying a contrast agent to the body tissue, wherein a duration of said countdown is determined at least in part based on the type of said contrast agent.

In some embodiments, said identifying comprises first identifying, in said at least one image, boundaries of said cervical tissue.

In some embodiments, said identifying is based, at least in part, on at least one of cervical tissue color and cervical surface texture.

In some embodiments, said identifying is based, at least in part, on executing one or more machine learning algorithms selected form the group consisting of convolutional neural network (CNN) classifiers and support vector machine (SVM) classifiers.

In some embodiments, said detecting is based, at least in part, on at least one of vaginal wall tissue color and vaginal surface texture.

In some embodiments, said detecting is based, at least in part, on one or more methods of feature extraction, wherein said feature is a ridges patterns of the surface of the vaginal wall.

In some embodiments, said determining is based, at least in part, on comparison of focus scores of (i) pixels in a region of said at least one image associated with said at least a portion of said vaginal wall, and (ii) pixels in another region of said at least one image associated with a central area of said TZ.

In some embodiments, said determining is based, at least in part, on a shape of a perimeter of said TZ.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a block diagram of an exemplary system for automated monitoring of the execution of a medical examination procedure involving the application of a contrast agent, according to an embodiment;

FIG. 2 illustrates generating a binary mask of a cervical region, according to an embodiment;

FIG. 3 illustrates generating a binary mask of a transformation zone of a cervix, according to an embodiment;

FIGS. 4A-4B illustrate portions of a speculum within a cervical image;

FIGS. 5A-5B illustrate a process for determining a positioning of vaginal walls within a cervical image, according to an embodiment;

FIG. 6 is a flowchart of a method for method for automated monitoring and detection of speculum positioning and/or visual obstruction by a vaginal wall in cervicography, according to an embodiment;

FIGS. 7A and 7B show multiple pairs of images: the left image in each pair is an input cervical image, and the right image is the output—according to experimental results;

FIG. 8 illustrates enhanced images of cervices and speculums; each row relates to a different cervical image, and columns show various added channels for each image—R, G, B, grayness, saturation, and value; and

FIG. 9 shows, on the left, a series of cervical RGB images each with a speculum, and on the right—the output images which show the speculum-related pixels (and all pixels, even of tissue, which are external to the speculum) in yellow.

### DETAILED DESCRIPTION

Disclosed herein are a system, method, and computer program product for the automated monitoring and detection of improper speculum positioning and/or visual obstruction by vaginal walls in cervicography.

In some embodiments, the present invention may be configured for automatically identifying and segmenting a region of interest (ROI) of the cervix in cervical images, using one or more segmentation methods. In some embodiments, the ROI is the transformation zone (TZ) of the cervix, an area in which almost all manifestations of cervical carcinogenesis occur.

In some embodiments, the present invention may further be configured for detecting and assessing a positioning of a speculum in the images relative to the ROI. For example, the present invention may be configured for detecting whether portions of the blades or other parts of a speculum obstruct or occlude any part of the TZ.

In some embodiments, the present invention may also be configured for detecting the vaginal walls in the images and determining whether any portion of the vaginal walls obstructs or occludes a portion of the ROI. For example, is some women who are obese or pregnant, or have had multiple vaginal deliveries, the vaginal walls become lax and redundant. In such cases, cervical images will be unsatisfactory since the view of the cervix is obstructed, unless the vaginal wall is pushed aside and supported. Pushing and supporting the vaginal wall may be achieved, e.g., using a vaginal wall retractor or by putting a condom over the blades of the speculum with the tip of the condom removed. See, e.g., Apgar, B., Brotzman,G., & Spitzer, M. (2008). 2nd ed. Colposcopy Principles and Practice: An Integrated Textbook and Atlas. Philadelphia, Pa.: W. B. Saunders.

In some embodiments, the present invention may then be configured for issuing appropriate alerts instructing a clinician conducting the cervicography to reposition the speculum and/or further push back the vaginal walls. Accordingly, the present invention may help to promote more consistent, accurate and reliable cervicography results. The increased accuracy and reliability of the results may offer a reduced risk of missing high grade disease, added reassurance in treatment decisions, and elimination of unnecessary treatments. In addition, by providing for greater consistency in imaging results, the present invention may facilitate greater use of computerized image evaluation applications, thereby reducing the reliance on medical experts and increasing overall efficiency.

In some embodiments, the present invention may be employed during cervical diagnostics and/or therapeutic procedures, such as colposcopy. In some embodiments, the present invention may be used in conjunction with a method for automated or partially-automated time-dependent image capture during colposcopy, based upon object recognition, such as the method disclosed by the present inventors in U.S. Provisional Patent Application No. 62/620,579 filed Jan. 23, 2018, which is incorporated herein by reference.

Cervicography is a method of cervical cancer screening which uses digital imaging of the cervix for enabling visual inspection of cervical tissue, e.g., before and after the application of a contrast agent which highlights pathological tissue. Cervicography can be used as an alternative method to Pap screening, especially in resource-poor regions where high-quality Pap screening programs often cannot be maintained because of inherent complexity and cost. The advances in digital imaging have enabled the acquisition of high quality cervix images at low cost, often using simple mobile devices, such as camera-equipped smartphones. These images can then be sent to experts, who may be located remotely, for evaluation. In some instances, automated or semi-automated image analysis applications may be employed to analyze the results. However, the accuracy of evaluation is greatly dependent on the training level of the clinician in following correct procedure for acquiring the images. Some of the issues which may arise in this regard, as noted above, include improper positioning of a speculum and/or partial visual obstruction by lax vaginal walls, both of which can occlude parts of the ROI in the images. In this regard, industry experts have estimated that clinicians may require performing between 25-100 cases with a preceptor, before their training is sufficient to ensure consistency in carrying out the cervicography (see, e.g., “Colposcopy can enhance your diagnostic skills,” Relias AHC Media, Jan. 1, 1998). However, in developing areas of the world, where examinations are sometimes conducted in field conditions, experienced or properly-trained staff may be difficult to recruit. Inconsistent or unreliable images, which are the result of an improperly conducted examination, may raise the issues of false positives and false negatives in the diagnosis. This may require routine double-checking by a medical expert, which runs counter to the purpose of using automated applications in the first place. In addition, poor diagnostic results may require the patient to return in order to be subjected again to a new colposcopy procedure, which again causes a waste of time and valuable resources.

Accordingly, a potential advantage of the present invention is in that it provides for a real-time, automated monitoring and detection of improper speculum positioning and/or visual obstruction by lax vaginal walls, thus promoting reliability and consistency in cervicography regardless of clinician training level. Since speculums of various materials and colors exist on the market, such as colorful plastic speculums and shiny metallic speculums, designing an algorithm to accommodate all them is not an easy task.

The following discussion will focus on cervicography. However, in addition to cervicography, the working principles of the present invention may be applied in other types of diagnostic and therapeutic treatments, which may benefit from improved consistency and reliability of visualization in imaging results.

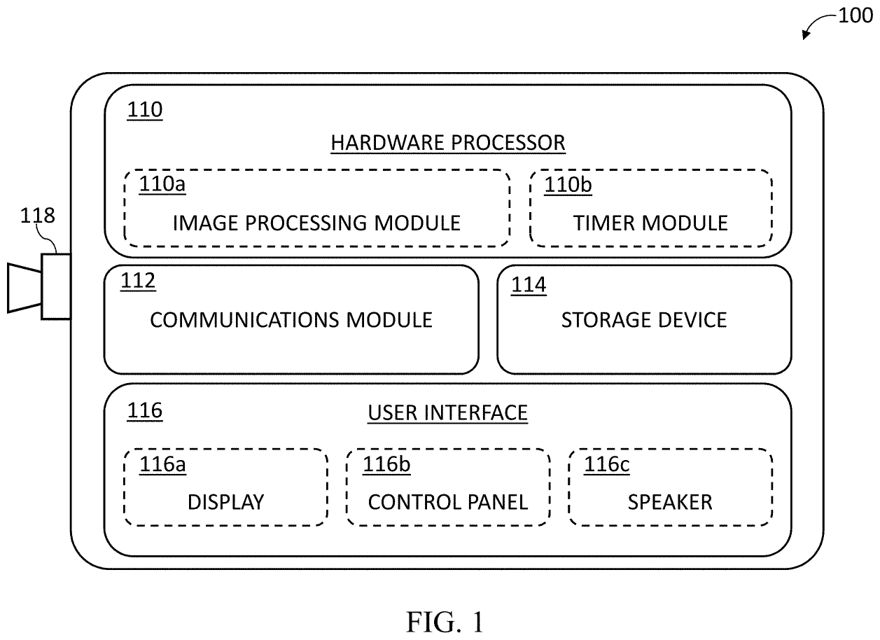

FIG. 1 is a block diagram of an exemplary system 100 for automated monitoring and detection of improper speculum positioning and/or visual obstruction by the vaginal walls in cervicography. System 100 as described herein is only an exemplary embodiment of the present invention, and in practice may have more or fewer components than shown, may combine two or more of the components, or a may have a different configuration or arrangement of the components. The various components of system 100 may be implemented in hardware, software or a combination of both hardware and software. In various embodiments, system 100 may comprise a dedicated hardware device, or may form an addition to or extension of an existing medical device, such as a colposcope.

In some embodiments, system 100 may comprise a hardware processor 110, communications module 112, memory storage device 114, user interface 116 and imaging device 118. System 100 may store in a non-volatile memory thereof, such as storage device 114, software instructions or components configured to operate a processing unit (also “hardware processor,” “CPU,” or simply “processor), such as hardware processor 110. In some embodiments, the software components may include an operating system, including various software components and/or drivers for controlling and managing general system tasks (e.g., memory management, storage device control, power management, etc.) and facilitating communication between various hardware and software components.

In some embodiments, non-transient computer-readable storage device 114 (which may include one or more computer readable storage mediums) is used for storing, retrieving, comparing, and/or annotating captured frames. Image frames may be stored on storage device 114 based on one or more attributes, or tags, such as a time stamp, a user-entered label, or the result of an applied image processing method indicating the association of the frames, to name a few.

The software instructions and/or components operating hardware processor 110 may include instructions for receiving and analyzing multiple frames captured by imaging device 118. For example, hardware processor 110 may comprise image processing module 110a, which receives one or more images and/or image streams from imaging device 118 and applies one or more image processing algorithms thereto. In some embodiments, image processing module 110a comprises one or more algorithms configured to perform object recognition and classification in images captured by imaging device 118, using any suitable image processing or feature extraction technique. For some embodiments, image processing module 110a can simultaneously receive and switch between multiple input image streams to multiple output devices while providing image stream processing functions on the image streams. The incoming image streams may come from various medical or other imaging devices. The image streams received by the image processing module 110a may vary in resolution, frame rate (e.g., between 15 and 35 frames per second), format, and protocol according to the characteristics and purpose of their respective source device. Depending on the embodiment, the image processing module 110a can route image streams through various processing functions, or to an output circuit that sends the processed image stream for presentation, e.g., on a display 116a, to a recording system, across a network, or to another logical destination. In image processing module 110a, the image stream processing algorithm may improve the visibility and reduce or eliminate distortion, glare, or other undesirable effects in the image stream provided by an imaging device. An image stream processing algorithm may reduce or remove fog, smoke, contaminants, or other obscurities present in the image stream. The types of image stream processing algorithms employed by the image stream processing module 110a may include, for example, a histogram equalization algorithm to improve image contrast, an algorithm including a convolution kernel that improves image clarity, and a color isolation algorithm. The image stream processing module 110a may apply image stream processing algorithms alone or in combination.

Image processing module 110a may also facilitate or recording operations with respect to an image stream. According to some embodiments, the image processing module 110a enables recording of the image stream with a voice-over or capturing of frames from an image stream (e.g., drag-and-drop a frame from the image stream to a window). Some or all of the functionality of the image processing module 110a may be facilitated through an image stream recording system or an image stream processing system.

Hardware processor 110 may also comprise timer module 110b, which may provide countdown capabilities using one or more countdown timers, clocks, stop-watches, alarms, and/or the like, that trigger various functions of system 100, such as image capture. Such timers, stop-watches and clocks may also be added and displayed over the image stream through user interface 116. For example, the timer module 110b may allow a user to add a countdown timer, e.g., in association with a surgical or diagnostic and/or other procedure. A user may be able to select from a list of pre-defined countdown timers, which may have been pre-defined by the user. In some variations, a countdown timer may be displayed on a display 116a, bordering or overlaying the image stream.

In some embodiments, system 100 comprises a communications module (or a set of instructions), a contact/motion module (or a set of instructions), a graphics module (or a set of instructions), a text input module (or a set of instructions), a Global Positioning System (GPS) module (or a set of instructions), voice recognition and/or and voice replication module (or a set of instructions), and one or more applications (or sets of instructions).

For example, a communications module 112 may connect system 100 to a network, such as the Internet, a local area network, a wide area network and/or a wireless network. Communications module 112 facilitates communications with other devices over one or more external ports, and also includes various software components for handling data received by system 100. For example, communications module 112 may provide access to a patient medical records database, e.g., from a hospital network. The content of the patient medical records may comprise a variety of formats, including images, audio, video, and text (e.g., documents). In some embodiments, system 100 may access information from a patient medical record database and provide such information through the user interface 116, presented over the image stream on display 116a. communications module 112 may also connect to a printing system configured to generate hard copies of images captured from an image stream received, processed, or presented through system 100.

In some embodiments, a user interface 116 of system 100 comprises a display monitor 116a for displaying images, a control panel 116b for controlling system 100, and a speaker 116c for providing audio feedback. In some variations, display 116a may be used as a viewfinder and/or a live display for either still and/or video image acquisition by imaging device 118. The image stream presented by display 116a may be one originating from imaging device 118. Display 116a may be a touch-sensitive display. The touch-sensitive display is sometimes called a “touch screen” for convenience and may also be known as or called a touch-sensitive display system. Touch-sensitive display may be configured to detect commands relating to activating or deactivating particular functions of system 100. Such functions may include, without limitation, image stream enhancement, management of windows for window-based functions, timers (e.g., clocks, countdown timers, and time-based alarms), tagging and tag tracking, image stream logging, performing measurements, two-dimensional to three-dimensional content conversion, and similarity searches.

Imaging device 118 is broadly defined as any device that captures images and represents them as data. Imaging devices may be optic-based, but may also include depth sensors, radio frequency imaging, ultrasound imaging, infrared imaging, and the like. In some embodiments, imaging device 118 may be configured to detect RGB (red-green-blue) spectral data. In other embodiments, imaging device 118 may be configured to detect at least one of monochrome, ultraviolet (UV), near infrared (NIR), and short-wave infrared (SWIR) spectral data. In some embodiments, imaging device 118 comprises a digital imaging sensor selected from the group consisting of complementary metal-oxide-semiconductor (CMOS), charge-coupled device (CCD), Indium gallium arsenide (InGaAs), and polarization-sensitive sensor element. In some embodiments, imaging device 118 is configured to capture images of a tissue sample in vivo, along a direct optical path from the tissue sample. In other embodiments, imaging device 118 is coupled to a light guide, e.g., a fiber optic light guide, for directing a reflectance and/or fluorescence from the tissue sample to imaging device 118. Imaging device 118 may further comprise, e.g., zoom, magnification, and/or focus capabilities. imaging device 118 may also comprise such functionalities as color filtering, polarization, and/or glare removal, for optimum visualization. Imaging device 118 may further include an image stream recording system configured to receive and store a recording of an image stream received, processed, and/or presented through system 100. In some embodiments, imaging device 118 may be configured to capture a plurality of RGB images, wherein imaging device 118 and/or image processing module 110a may be configured for changing the ratio of individual RGB channels, for example, by amplifying at least one of the green channel and the red channel.

System 100 may further comprise, e.g., light collection optics; beam splitters and dichroic mirrors to split and direct a desired portion of the spectral information towards more than one imaging device; and/or multiple optical filters having different spectral transmittance properties, for selectively passing or rejecting passage of radiation in a wavelength-, polarization-, and/or frequency-dependent manner.

In some embodiments, system 100 includes one or more user input control devices, such as a physical or virtual joystick, mouse, and/or click wheel. In other variations, system 100 comprises one or more of a peripherals interface, RF circuitry, audio circuitry, a microphone, an input/output (I/O) subsystem, other input or control devices, optical or other sensors, and an external port. System 100 may also comprise one or more sensors, such a proximity sensors and/or accelerometers. Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments.

In some embodiments, system 100 is mounted on a stand, a tripod and/or a mount, which may be configured for easy movability and maneuvering (e.g., through caster wheels). In some embodiments, the stand may incorporate a swingarm or another type of an articulated arm. In such embodiments, imaging device 118 may be mounted on the swingarm, to allow hands-free, stable positioning and orientation of imaging device 118 for desired image acquisition. In other embodiments, system 100 may comprise a portable, hand-held colposcope.

In some embodiments, a system such as system 100 shown in FIG. 1 is configured for capturing an initial one or more reference images and/or begin a continuous video stream of image frames of the cervix, using, e.g., imaging device 118. System 100 may then be configured for performing segmentation on one or more of the acquired cervical images, to automatically identify and delineate the cervical borders within the images.

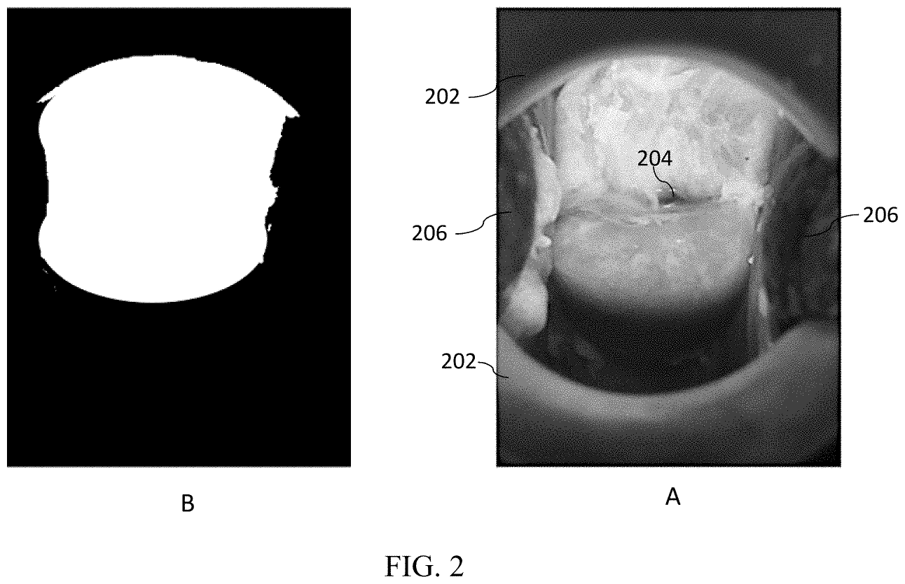

FIG. 2, in panel A, shows an image of a cervix captured through an opening of a speculum, wherein the cervix region appears in the image as a relatively pink region (shown in grayscale) located around the image center. The cervix region is shown framed within visible portions 204 of a speculum, and vaginal walls 206.

In some embodiments, system 100 may apply a specified color filter, such as a filter which selects for pink/red/white, for color-based identification of the cervix, based on normal cervical tissue color. In some embodiments, a specified color model, such as the L*a*b* Color space, may be used. Thus, for example, the red component of the cervix image may be filtered, and a thresholding operation may be applied to the resulting image, to generate a binary mask of the cervical region (panel B in FIG. 2).

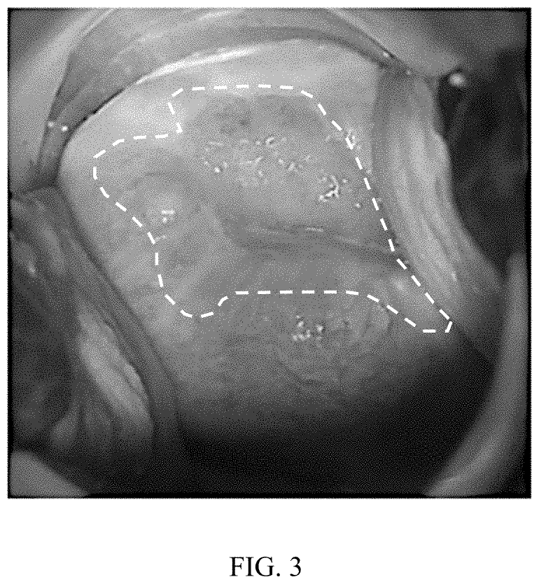

With reference to FIG. 3, after system 100 has identified the cervical region in the image stream, system 100 may be configured for further identifying a transformation zone (TZ) within the cervical area, which is the main region of interest (ROI) for purposes of detecting pathological tissue. The TZ (demarcated by a dashed line in FIG. 3) is a region of the cervix where the columnar epithelium has been replaced and/or is being replaced by new metaplastic squamous epithelium. The TZ corresponds to the area of cervix bound by the original squamo-columnar junction (SCJ) at the distal end and proximally by the furthest extent that squamous metaplasia has occurred as defined by the new squamo-columnar junction. In premenopausal women, the TZ is fully located on the ectocervix, and may move radially back and forth over the course of the menstrual cycle. After menopause and through old age, the cervix shrinks with the decreasing levels of estrogen. Consequently, the TZ may move partially, and later fully, into the cervical canal. Identifying the transformation zone is of great importance in colposcopy, as almost all manifestations of cervical carcinogenesis occur in this zone.

Additionally or alternatively, system 100 may be configured to identify the SCJ within the cervical area. The SCJ is defined the junction between the squamous epithelium and the columnar epithelium. Its location on the cervix is variable. The SCJ is the result of a continuous remodeling process resulting from uterine growth, cervical enlargement and hormonal status.

In some embodiments, system 100 may employ one or more known computer vision methods for identifying and segmenting the cervix and identifying the TZ, such as the algorithm developed in connection with the Kaggle, Inc. competition hosted by Intel® and the present applicant (see www.kaggle.com/c/intel-mobileodt-cervical-cancer-screening). This algorithm, which is incorporated herein by reference, is trained to classify cervix types in images (e.g., types I, II, and III), based on the location of the TZ in the image, and thus useful for segmenting the TZ in cervical images.

In other instances, image processing module 110a may employ a dedicated object recognition and classification application for identifying one or more regions, features, and/or objects in the image. The application may first conduct feature-level detection, to detect and localize the objects in the image, and then perform decision-level classification, e.g., assign classifications to the detected features, based on the training of the application. The application may use machine learning algorithms to train it to identify objects in given categories and subcategories. For example, the application may use one or more machine learning algorithms, such as a support vector machine (SVM) model and/or a convolutional neural network (CNN). SVMs are supervised learning models that analyze data used for classification and regression analysis. Given a set of training examples, each marked as belonging to one or the other of two categories, an SVM training algorithm builds a model that assigns new examples to one category or the other, making it a non-probabilistic binary linear classifier. A SVM model is a representation of the examples as points in space, mapped so that the examples of the separate categories are divided by a clear gap that is as wide as possible. New examples are then mapped into that same space and predicted to belong to a category based on which side of the gap they fall. CNNs are a class of deep, feed-forward artificial neural networks, most commonly applied to analyzing visual imagery. A CNN algorithm may be trained by uploading multiple images of items in the category and subcategories of interest. For example, the CNN algorithm may be trained on a training set of properly-labeled relevant images, which may include multiple cervical images of different sizes, taken from different angles and orientations, under varying lighting conditions, and with different forms of occlusion and interferences. The CNN algorithm may also be provided with a set of negative examples, to further hone the classifier training. The CNN algorithm applies a convolutional process to classify the objects in each training image in an iterative process, in which in each iteration, (i) a classification error is calculated based on the results, and (ii) the parameters and weights of the various filters used by the CNN algorithm are adjusted for the next iteration, until the calculated error is minimized. This means that the CNN algorithm can be optimized to recognize and correctly classify images from the labelled training set. After training completes, when a new image is uploaded to the CNN algorithm, the CNN algorithm applies the same process using the parameters and weights which have been optimized for the relevant category, to classify the new image (i.e., assign the correct labels to objects identified therein) with a corresponding confidence score.

For example, image processing module 110a may first employ a CNN algorithm trained to detect the cervix. In successful experiments conducted by the inventors, RetinaNet (Tsung-Yi Lin, Priya Goyal, Ross Girshick, Kaiming He, Piotr Dollár, “Focal Loss for Dense Object Detection,” in IEEE International Conference on Computer Vision (ICCV), 2017, pp. 2980-2988) has been used to detect the location of the cervix in images, by training it on manually-annotated images with cervix bounding boxes. Next, image processing module 110a may use a semantic segmentation CNN to segment the TZ and/or the SCJ. In successful experiments conducted by the inventors, U-Net (Olaf Ronneberger, Philipp Fischer, Thomas Brox, “U-Net: Convolutional Networks for Biomedical Image Segmentation,” Medical Image Computing and Computer-Assisted Intervention (MICCAI), Springer, LNCS, Vol. 9351: 234-241, 2015) has been used to segment the TZ and the SCJ, by training it on manually-annotated images in which the TZ and the SCJ, respectively, are marked. Different instances of a trained U-Net may be used to segment the TZ and the SCJ, so two runs of the U-Net may be required if segmentation of bot the TZ and the SCJ is desired.

Optionally, the algorithm used to segment the TZ and the SCJ may take into account ordinal arrangement of concepts in the image. Since the SCJ and TZ hold a spatial arrangement with respect to the external os and the cervix itself, several additional channels may be included as features to the CNN's input. This promotes the creation of spatially-aware convolutional kernels. For example, one or more of the following additional channels may be included:
Distance from the TZ/SCJ to the image center.
Distance from the TZ/SCJ to the external os.
Distance from the TZ/SCJ to the cervix boundary.
Grayness of the TZ/SCJ.
Saturation and value channels (from an HSV color space of the image) of the TZ/SCJ.
RGB channel values of the TZ/SCJ.
Additional feature spaces may cover the AW (aceto-whitened) filters proposed in the literature. For a survey of such methods, see Fernandes, Kelwin, Jaime S. Cardoso, and Jessica Fernandes, “Automated methods for the decision support of cervical cancer screening using digital colposcopies.” IEEE Access 6 (2018): 33910-33927.

The distance to the external os requires an additional model that, given a cervical image, returns the coordinates of the os. Methods to predict the position of the os are presented in the aforementioned paper. Most of the methods to detect the os consider zones with concavities (the os is typically darker than the rest of the cervix). Alternatively, a regression-based CNN or a patch-based binary CNN may be trained to detect the external os location.

The distance to the cervix boundary requires an additional model that segments the cervix. Various deep architecture for image segmentation, such the ones discussed above, may be used for this.

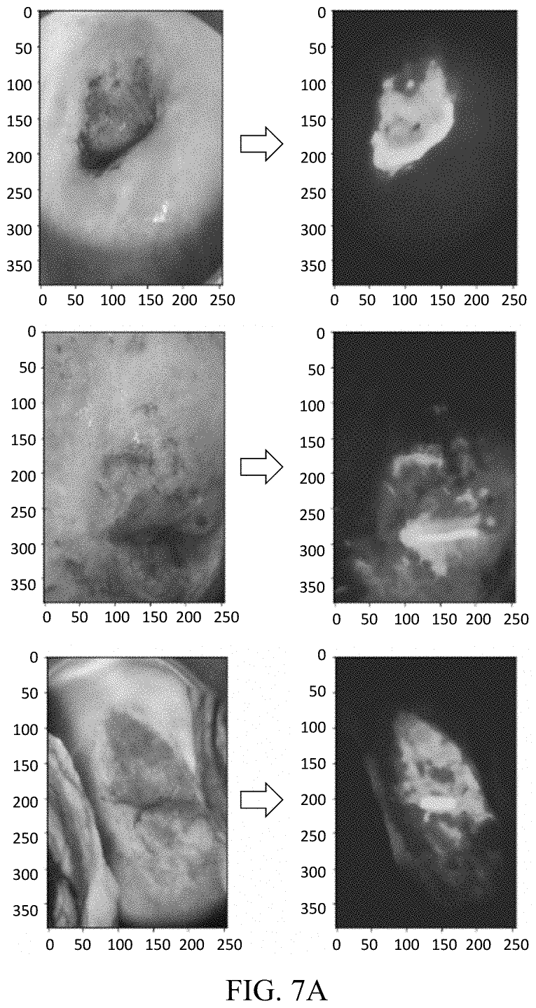

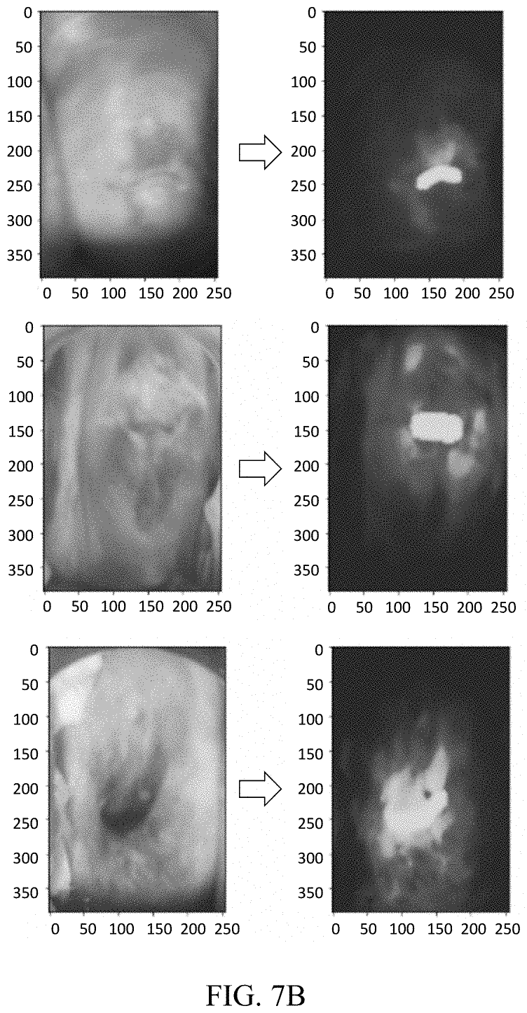

In successful experiments conducted by the inventors, the inclusion of the distances to the external os and the cervix boundary improved the pixelwise accuracy for the SCJ by 2%, the Dice's coefficient from 44% to 60% and the pixelwise ROC AUC from 80% to 98%. FIGS. 7A and 7B show the results of these experiments. Multiple pairs of images are shown—the left image in the pair is the input cervical image, and the right image is the output, in which the detected speculum is painted dark blue.

An additional approach for detection of the cervix may include enhancing the image with one or more additional channels that typically correlate with speculum pixels. For example:
For metallic, shiny speculums, grayness of pixels may be indicative of the speculum. Grayness is defined herein as the ratio between the minimum and maximum channels per pixel:

$$\frac{min ( R , G , B ) + 1}{max ( R , G , B ) + 1}$$

For plastic, colored speculums, saturation and value from the HSV color space may be indicative of the speculum. Namely, those pixels with relatively high saturation and value are likely associated with a speculum.
Other channels may be considered, such as distance to the image borders (a speculum is usually found close to image borders), relative grayness with respect to the remaining pixels in the image, etc.

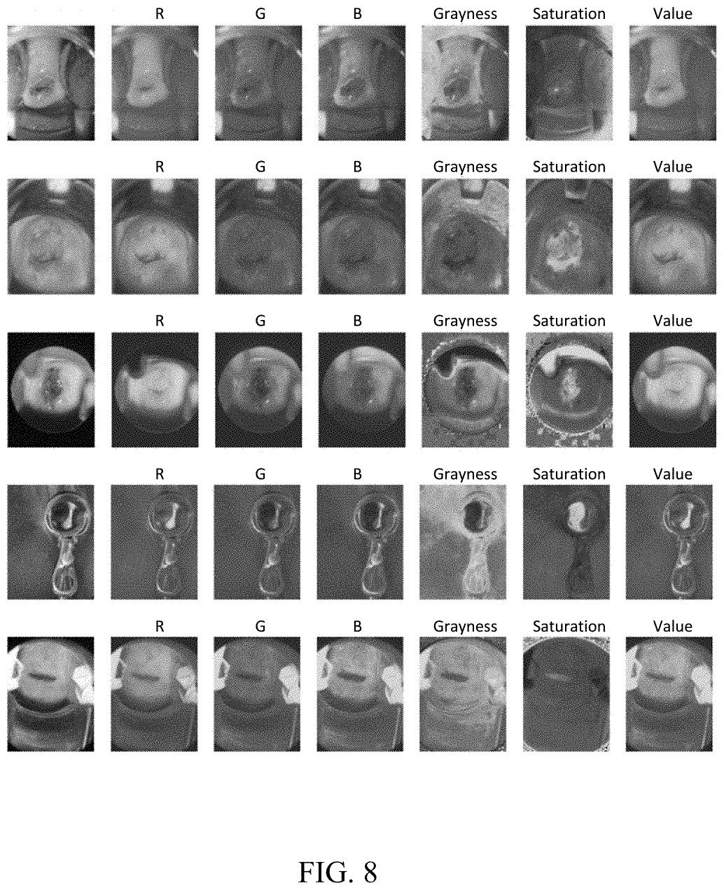

FIG. 8 illustrates enhanced images of cervices and speculums. Each row in the figure relates to a different image. Columns show various added channels for each image—R, G, B, grayness, saturation, and value.

Successful experiments conducted by the inventors have confirmed that the addition of the aforementioned additional channels yields excellent detection of speculums in cervical images. A U-net architecture was trained with four blocks of convolutional layers (two consecutive layers per block) on each part of the encoder-decoder architecture. A dataset with 1480 labeled images was split into training-validation-test partitions following the traditional 60-20-20 distribution. A performance of 82.41% Dice's coefficient and a pixelwise ROC AUC of 93.22% was achieved.

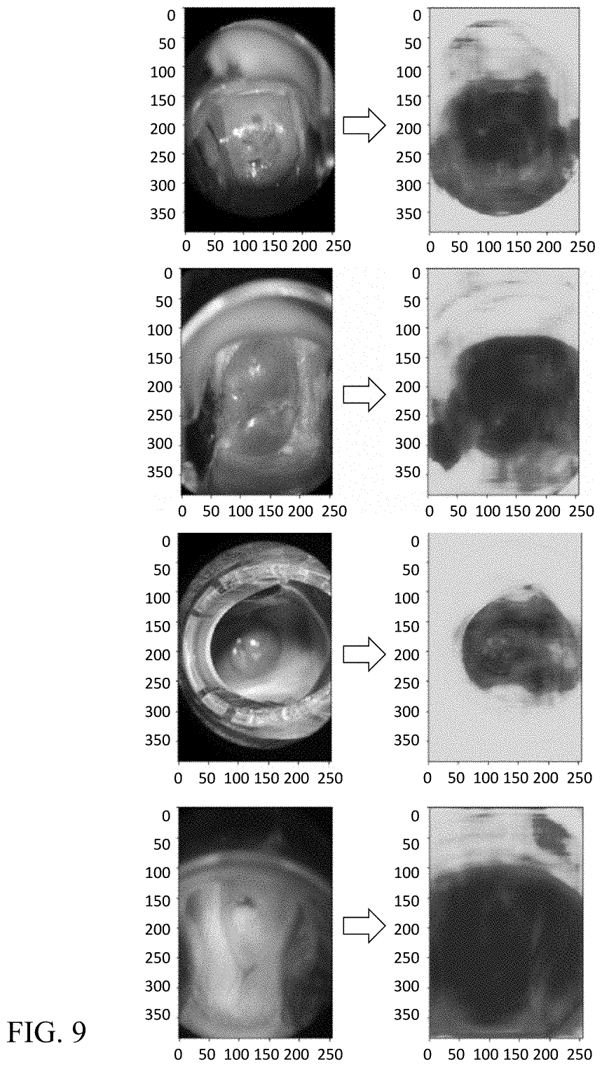

FIG. 9 shows, on the left, a series of cervical RGB images with a speculum, and on the right—the output images which show the speculum-related pixels (and all pixels, even of tissue, which are external to the speculum) in yellow.

Optionally, in order to remove small pixel blobs that may be returned by the encoder-decoder network, noisy over/under-detections may be removed using morphological operators and connected components analysis.

The estimation of the speculum location may be further improved by considering continuous sequences of images (frames of a video stream) and by aggregating the speculum masks (marked locations of the speculum) estimated by the CNN on each one of the frames. Alternatively, if working with still images or with temporally-distant images, multiple estimations from synthetically augmented images be aggregated.

In some embodiments, system 100 may then be configured for identifying the visible portions of a speculum in the image. Namely, the initial segmentation of the cervix, the TZ and/or the SCJ may be utilized to narrow down the region in the image where the speculum is looked for.

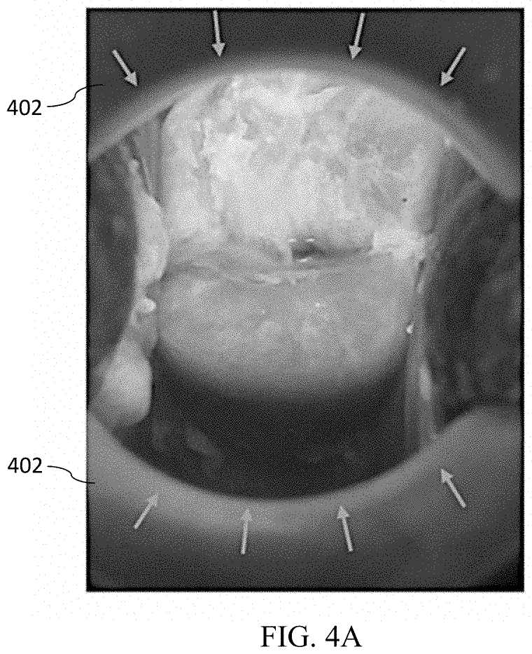

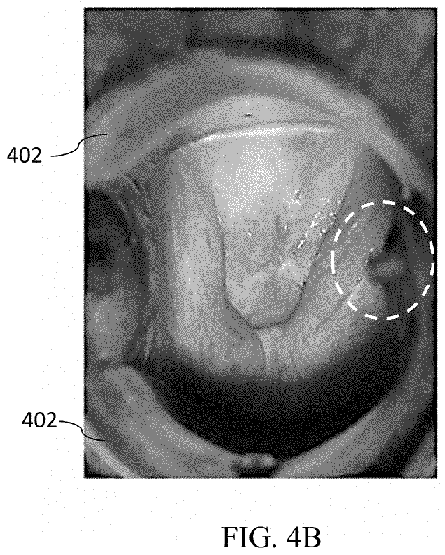

With reference to FIG. 4A, during cervicography, a bi-valved vaginal speculum or a similar device typically s inserted into the vagina to afford an internal view of the vaginal wall and the cervix. Images, such as those taken by imaging device 118, are then taken through an opening of the speculum, such that portions 402 of the speculum blades are at least partially visible in the image. When incorrectly positioned, portions 402 may at least partially occlude the TZ in the image. Accordingly, it is crucial to identify portions 402 in the image, as well as their relative position with respect to the TZ, to ensure full visibility of the TZ in cervical images.

As can be seen in FIG. 4A, the visible portions 402 of the speculum blades typically appear as curved lips (marked by multiple arrows) enclosing the cervix on two sides. Accordingly, in some embodiments, System 100 may be configured for identifying the curvature of portions 402, using an appropriate feature extraction method, such as a Hough Transform, the ‘fitellipse’ technique (see https://docs.opencv.org/3.4/de/dc7/fitellipse_8cpp-example.html), and/or another similar method.

Once the curvature of portions 402 has been identified in the image, system 100 may be configured for segmenting portions 402 in the image, based, e.g., on detecting a color associated with portions 402. It should be noted that specula blades and other parts typically are made of a single material (e.g., stainless steel, plastic) and thus exhibit a uniform color (e.g., metallic silver/grey, clear plastic, blue plastic, etc.). Using known segmentation methods, portions 402 may then be segmented and identified in the images in their entirety.

Following the identification and segmentation of the cervical region, TZ, and visible speculum portions 402, as discussed above, system 100 may further be configured for determining a positional relationship between portions 402 and the TZ. In some embodiments, system 100 is configured for determining a distance parameter between boundaries of the TZ (previously identified in the image) and speculum portions 402, wherein a close proximity of speculum portions 402 to the TZ may affect image sharpness at least partially. When system 100 determines that the TZ boundaries and portions 402 are in close proximity and/or contacting engagement, system 100 may further be configured for calculating a focus score for pixels in the TZ boundary areas. These focus scores may then be compared to focus scores of pixels in other areas of the TZ, wherein a lower focus score for pixels in the boundaries areas may lead to a determination of an incorrect positioning of the speculum in relation to the TZ. Following a determination of incorrect positioning, system 100 may be configured for issuing an alert to the clinician performing the cervicography to reposition the speculum. The alert to the clinician may be, e.g., a visual, auditory, and/or verbal alert, communicated through display 116a and/or speaker 116c. Following the issuance of the alert, system 100 may be configured for re-evaluating the positioning of the speculum, before issuing an appropriate indication to the clinician to proceed with the procedure.

In some embodiments, system 100 may be configured for calculating a focus score based on the method proposed by the present inventors and others in M. Jaiswal et al., “Characterization of cervigram image sharpness using multiple self-referenced measurements and random forest classifiers,” Proc. SPIE 10485, Optics and Biophotonics in Low-Resource Settings IV, 1048507 (13 Feb. 2018); doi: 10.1117/12.2292179; and/or the method proposed in S. Pertuz et al., “Analysis of focus measure operators for shape-from-focus”, Pattern Recognition 46 (2013) 1415-1432, both of which are incorporated herein by reference.

With reference to FIG. 4B, in other embodiments, system 100 may be configured for determining whether parts of a speculum occlude or obstruct the cervix field of view. FIG. 4B shows a cervical image in which a speculum portion (marked by a dashed circle) blocks part of the TZ, thereby restricting the visibility of TZ tissue in that area. System 100 may be configured for using the TZ mask previously generated, by dilating and eroding the mask around a region near the boundary of the TZ, to create a secondary mask of pixels localized near the boundary TZ/speculum. Following this, system 100 may determine whether speculum portions are visible within the ROI, and an appropriate alert may be issued to the clinician performing the examination to reposition the speculum. As noted above, the alert to the clinician may be, e.g., a visual, auditory, and/or verbal alert, communicated through display 116a and/or speaker 116c.

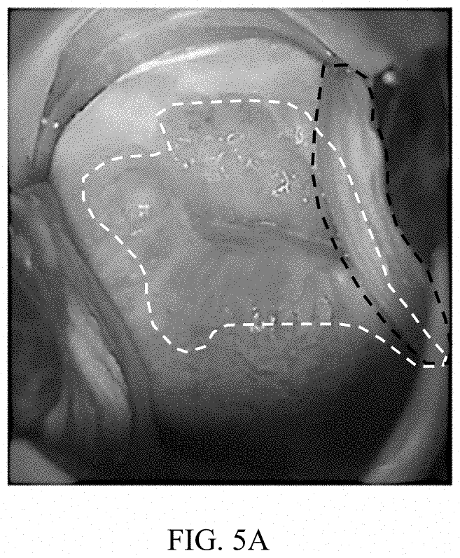

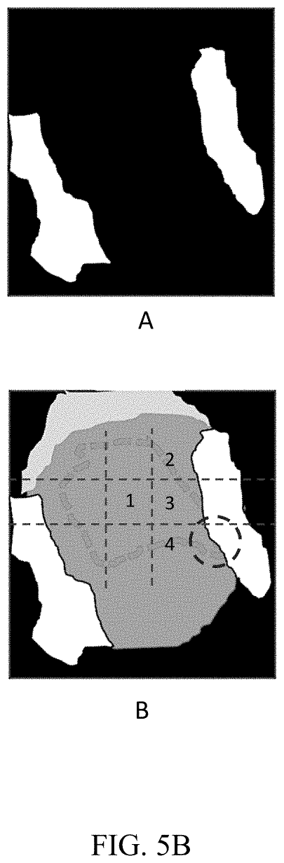

With reference to FIG. 5A, in some embodiments, system 100 may be further configured for identifying vaginal walls in the cervical image, when parts of laxed and/or prolapsed vaginal wall (delineated by a black dashed line) visually obstructs at least a portion of the TZ (delineated by a white dashed line).

With reference to FIG. 5B, in some embodiments, system 100 may first be configured for creating a mask of pixels that are not in the TZ (white areas in panel A in FIG. 5B). It should be noted that cervical tissue and vaginal wall tissue may exhibit different shades of pink. Accordingly, segmenting the vaginal walls from the cervical tissue may be based, e.g., on surface texture and/or pixel color-based filtering. System 100 may be then configured for dividing the TZ into, e.g., a 3×3 grid, and calculating a reference focus score for a center square of the grid (e.g., square 1 in panel B). System 100 may then be configured for calculating a focus score for squares to the left and right of central square 1 (e.g., squares 2, 3, 4 in panel B). System 100 may then be configured for locating a boundary region within the TZ mask where focus scores for pixels begin to decline in comparison to the reference focus scores calculated for square 1 (e.g., the region demarcated by a circle in panel B). System 100 may then determine that pixels within the TZ mask which exhibit a focus score lower than the reference focus score belong to a vaginal wall portion.

In other embodiments, system 100 may be configured for detecting vaginal walls in the cervical image, e.g., by identifying one or more features specific to the vaginal wall surface structure. For example, system 100 may be configured for detecting periodicity in ridges of the vaginal wall, e.g., using known spatial transform methods, such as a Fourier Transform. When applied in the horizontal direction, such spatial transform methods may enable detecting specific spatial frequency bands which manifest in the vaginal walls, but not in the cervical tissue. In other embodiments, additional and/or other spatial transforms may be used to identify specific surface features of the vaginal walls.

In yet other embodiments, system 100 may be configured for assessing a positional relationship between the TZ and the vaginal walls based, at least on part, on the shape of the TZ mask. For example, a TZ which is not obstructed by portions of the vaginal walls should exhibit a generally convex perimeter shape, with no significant concavities. Accordingly, a detected concavity within the TZ mask (e.g., an hourglass shape) may be an indication of visual obstruction by lax vaginal walls.

In the above embodiments, following the detection of vaginal walls in the cervical image, system 100 may be configured for determining a positional relationship between the vaginal walls and the TZ. If system 100 determines that a gap or discontinuity exists within the TZ mask due to obstruction by the vaginal walls, system 100 may be configured for issuing an appropriate alert to the user. In some embodiments, such an alert to the user may include instructions to further open the vaginal walls, e.g., using an appropriate medical accessory. In some cases, lax or prolapsed vaginal walls may be pushed aside using, e.g., a vaginal wall retractor. In other cases, the clinician may slide a condom (with the condom tip removed) over the blades of the speculum. Following the issuance of the alert, system 100 may be configured for re-evaluating the positioning of the vaginal walls, before issuing an appropriate indication to the clinician to proceed with, e.g., a diagnostic and/or therapeutic procedure.

In some embodiments, following a determination by system 100 regarding correct speculum positioning and/or removal of vaginal walls visual obstruction in the cervical image, system 100 may then be configured for automated capturing of a plurality of images of the cervix. The plurality of images may be captured by system 100 in specified successive time instances, and/or from a continuous video stream. In some cases, some of the plurality of images will be captured before and after application of a contrast agent, to document both (i) every spatial point of the tissue area under analysis, and (ii) temporal progression of the alterations in the tissue over time.

In some embodiments, system 100 may be configured for automated image capture upon detecting correct speculum positioning and/or absence of visual obstruction by vaginal walls. In some embodiments, system 100 may then be configured for instructing the clinician to release and/or relax the vaginal walls and apply a contrast agent to the cervical tissue. In some embodiments, system 100 may then be configured for (i) alerting the clinician to re-open the vaginal walls, (ii) detecting, e.g., a swab in the field of view of imaging device 118 which indicates the application by the clinician of the contrast agent, and (ii) automatically capturing one or more images upon the expiration of a specified countdown following the detection of the swab.

As noted above, in some embodiments, the present invention may incorporate the method for automated time-dependent image capture based upon object recognition disclosed by the present inventors in U.S. Provisional Patent Application No. 62/620,579 filed Jan. 23, 2018, which is incorporated herein by reference. The identification of the presence of a swab in the field of view of the imaging device may be used as an indication of the application of the contrast agent, thus triggering one or more predetermined countdowns for image capture, e.g., using timer module 110b. For example, system 100 may be configured to capture an image at 120 seconds after commencement application of the contrast agent, or, e.g., a series of images at time intervals 30, 60, and 90 seconds, respectively. Optionally, system 100 may acquire a continuous or a time-lapse video of the entire procedure, where time intervals may be indicated for individual frames within the video.

In some embodiments, system 100 may be configured for capturing a sequential or periodic series of images according to predetermined protocols; capturing a sequence (or burst) of images around the expiration of the counter (e.g., slightly before and/or after); or capturing multiple sequential images and selecting one or more images from the sequence based on an image quality parameter. In the context of a continuous stream of images or a video acquired by imaging device 118, processed by system 100 and displayed of display 116a, capturing an individual image at a specific time point may involve, e.g., capturing and saving one or more image frames corresponding to specific time points, and/or applying suitable captioning and/or annotation to one or more image frames in a video, representing a snapshot of the image stream at the desired time point. The captured image(s) may then be stored, e.g., in an image database on storage device 114. For example, system 100 may be configured to mark or annotate stored image with necessary information or metadata, such as patient or user name, date, location, and other desired information. In some embodiments, system 100 may generate automatic electronic patient records comprising all images captured over multiple procedures in connection with a patient or user, to enable, e.g., post-exam reviews, longitudinal tracking of changes over time, and the like. Such records may be stored in a database on storage device 114. In some variations, patient records may be EMR- (electronic medical records) compatible, to facilitate identifying patients who are due for preventive visits and screenings, and monitor how patients measure up to certain parameters. EMR records may then be shared over a network through, e.g., communications module 112.

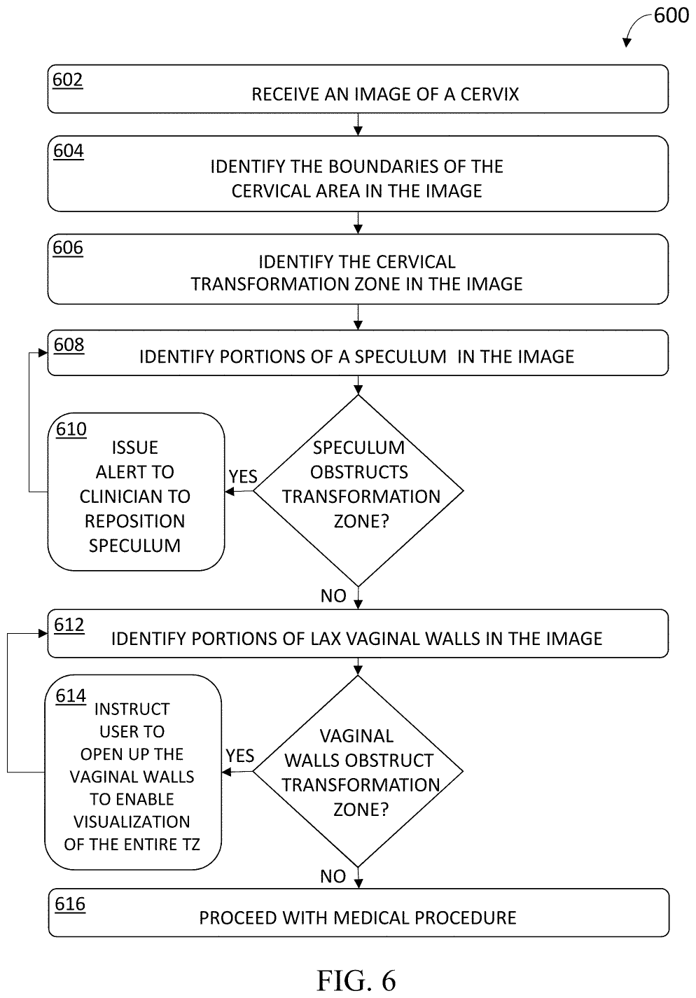

FIG. 6 is a flowchart illustrating an exemplary method 600, according to certain embodiments of the present disclosure. The steps of method 600 are described herein with reference to the medical diagnostic procedure of colposcopy.

At 602, an imaging device, such as imaging device 118 of system 100 in FIG. 1, is positioned so as to gain a view of a cervix, e.g., through the opening of a vaginal speculum or similar device providing an internal view of the vaginal wall and the cervix. Imaging device 118 then transmits a series of images and/or a video stream of an area of the cervix under observation. In some instances, one or more baselines images of the cervix are captured at this stage, to use as future reference. In some embodiments, the image stream acquired by imaging device 118 is displayed live on display 116a. Image processing module 110a may apply continuously visual recognition algorithms to the images streamed by imaging device 118.

At 604, system 100 identifies the boundaries of the cervix in the image stream, based, at least in part, on color parameters of the cervical tissue. System 100 creates a binary mask of the cervical area as applied to the image stream.

At 606, system 100 identifies the boundaries of a TZ of the cervix within the images.

At 608, system 100 identifies one or more portions of the blades of the vaginal speculum within the images, and determines a position of the speculum in relation to the TZ.

At 610, if system 100 determines that the speculum obstructs at least part of the TZ, system 100 issues an appropriate alert to the clinician to reposition the speculum. System 100 may then repeat steps 608-610 until it is determined that the speculum is correctly positioned.

At 612, system 100 identifies portions of laxed vaginal walls in the images and determines whether the vaginal walls obstruct, at least in part, areas of the TZ.

At 614, if system 100 determines that the vaginal walls obstruct at least part of the TZ, system 100 issues an appropriate instructions to the clinician to further open the vaginal walls. System 100 may then repeat steps 612-614 until it is determined that the vaginal walls no longer visually obstruct the TZ.

Finally, at 616, system 100 may indicate to the clinician that the examination may proceed to the next steps.

In an embodiment, the detection of the speculum in a cervical image may be utilized to indicate to the user that the image is incorrectly focused on the vulva, so that the user may bring the imaging device closer to the patient, increase optical zoom (if this feature exists in the imaging device), or instruct the imaging device to focus on the cervix instead of the vulva (e.g., by interacting with a graphical user interface of the device). Incorrect focusing is a common problem with imaging devices that have a relatively shallow depth of field, such as smart phones and other portable computing devices that include a camera. Sometimes, an add-on lens assembly is used with such portable computing devices, mainly for magnification purposes. One example is the EVA System by MobileODT Ltd., of Tel Aviv, Israel. The shallow depth of field prevents simultaneously focusing on objects at different distances from the camera, even if the distance between the objects is not that far (as is the distance between the vulva and the cervix).

Accordingly, after the speculum has been detected in an image, system 100 may evaluate whether the vulva is also depicted in the image (externally to the speculum), and if so—how much of it is in the image. For example, the area depicting the vulva in the image may be quantified as a ratio between the area depicting the cervix and the area depicting anything external to the speculum. As another example, the area depicting vulva in the image may be calculated as a ratio between the area depicting the speculum and the area depicting anything external to the speculum. A further example is a predefined pixel count of anything external to the speculum.

If the area depicting the vulva exceeds a predefined threshold, then a further check may be made to determine if the imaging device has been focused on the vulva instead of on the cervix: the amount of contrast of the vulva may be compared with the amount of contrast of the cervix, such as by comparing an AUC of a contrast histogram of each of these regions or by using any other method known in the art for contrast evaluation. The region with the higher contrast is very likely the region on which the imaging device has focused. Thus, if the higher contrast is of the area depicting the vulva, then the user may be immediately instructed to perform one or more of the above-mentioned steps to correctly focus on the cervix. Immediate, real-time alert to the user is very important, and may prevent the user from the continuing to record a cervicography session with incorrect focus—whose imagery will later not be useful for clinical analysis of the cervix.

As an alternative to contrast comparison, any other method known in the art for evaluating which region in an image is in focus may be used.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The description of a numerical range should be considered to have specifically disclosed all the possible subranges as well as individual numerical values within that range. For example, description of a range from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A method comprising:
capturing at least one image of a cervical tissue in-vivo;
identifying a region of interest (ROI) in said cervical tissue within said at least one image;
detecting at least a portion of a vaginal speculum, and at least a portion of a vaginal wall, within said at least one image;
determining a position of said portion of said vaginal speculum, and a position of said portion of said vaginal wall, relative to said ROI; and
issuing an alert when said determining indicates that at least one of said portion of said vaginal speculum and said portion of said vaginal wall obstruct, at least in part, said ROI in said at least one image,
wherein said alert directs a clinician to reposition said vaginal speculum, or to push aside said portion of said vaginal wall, or both.

2. (canceled)

3. The method of claim 1, further comprising repeating iteratively said detecting, determining, and issuing until said determining indicates that neither said portion of said vaginal speculum nor said portion of said vaginal wall obstruct said ROI in said at least one image.

4. The method of claim 3, further comprising capturing one or more images upon said indicating.

5. The method of claim 4, wherein said identifying comprises first identifying, in said at least one image, boundaries of said cervical tissue.

6. The method of claim 4, wherein:
said identifying is based, at least in part, on at least one of one of cervical tissue color and cervical surface texture.

7. The method any claim 4, wherein said identifying is based, at least in part, on executing one or more machine learning algorithms selected from the group consisting of convolutional neural network (CNN) classifiers and support vector machine (SVM) classifiers.

8. The method of claim 4, wherein:
said detecting of said portion of said vaginal speculum is based, at least in part, on one or more methods of feature extraction, wherein said feature is an arch-like end portion of a blade of said vaginal speculum; and
said detecting of said portion of said vaginal wall is based, at least in part, on one or more methods of feature extraction, wherein said feature is a ridges patterns of the surface of the vaginal wall.

9. The method of claim 4, wherein said determining of the position of said portion of said vaginal speculum is based, at least in part, on a comparison of focus scores of (i) pixels in a region of said at least one image associated with said at least a portion of said vaginal speculum, and (ii) pixels in another region of said at least one image associated with said ROI.

10. The method of claim 4, wherein said determining is based, at least in part, on morphologically dilated versions of regions of said at least one image, wherein a first one of said regions is associated with said at least a portion of said vaginal speculum, and wherein a second one of said regions is associated with said at least a portion of said vaginal wall.

11. The method of claim 1, further comprising issuing an alert to direct a focus of the capturing to the cervix if the focus of the at least one image is determined to be on a vulva.

12. The method of claim 1, wherein the ROI is a transformation zone of the cervix.

13-26. (canceled)

27. A system comprising:
at least one hardware processor; and
a non-transitory computer-readable storage medium having stored thereon program instructions, the program instructions executable by the at least one hardware processor to:
operate an imaging device to capture at least one image of a cervical tissue in-vivo,
identify a region of interest (ROI) in said cervical tissue within said at least one image,
detect at least a portion of a vaginal speculum, and at least a portion of a vaginal wall, within said at least one image,
determine a position of said portion of said vaginal speculum, and a position of said portion of said vaginal wall, relative to said ROI, and
issue an alert when said determining indicates that at least one of said portion of said vaginal speculum and said portion of said vaginal wall obstruct, at least in part, said ROI in said at least one image,
wherein said alert directs a clinician to reposition said vaginal speculum, or to push aside said portion of said vaginal wall, or both.

28. (canceled)

29. The system of claim 27, wherein said instructions further comprise repeating iteratively said detecting, determining, and issuing until said determining indicates that neither said portion of said vaginal speculum nor said portion of said vaginal wall obstruct said ROI in said at least one image.

30. The system of claim 27, wherein said instructions further comprise operating said imaging device to capture one or more images upon said indicating.

31. The system of claim 30, wherein said identifying comprises first identifying, in said at least one image, boundaries of said cervical tissue.

32. The system of claim 30, wherein said identifying is based, at least in part, on at least one of one of cervical tissue color and cervical surface texture.

33. The system of claim 30, wherein said identifying is based, at least in part, on executing one or more machine learning algorithms selected from the group consisting of convolutional neural network (CNN) classifiers and support vector machine (SVM) classifiers.

34. The system of claim 30, wherein:
said detecting of said portion of said vaginal speculum is based, at least in part, on one or more methods of feature extraction, wherein said feature is an arch-like end portion of a blade of said vaginal speculum; and
said detecting of said portion of said vaginal wall is based, at least in part, on one or more methods of feature extraction, wherein said feature is a ridges patterns of the surface of the vaginal wall.

35. The system of claim 30, wherein said determining of the position of said portion of said vaginal speculum is based, at least in part, on a comparison of focus scores of (i) pixels in a region of said at least one image associated with said at least a portion of said vaginal speculum, and (ii) pixels in another region of said at least one image associated with said ROI.

36. The system of claim 30, wherein said determining is based, at least in part, on morphologically dilated versions of regions of said at least one image, wherein a first one of said regions is associated with said at least a portion of said vaginal speculum, and wherein a second one of said regions is associated with said at least a portion of said vaginal wall.

37.-52. (canceled)
